(19) United States
(12) Patent Application Publication
(10) Pub. No.: US 2026/0321362 A1
(43) Pub. Date: Oct. 15, 2026

(54) MACHINE LEARNING BASED COMPOUND SCREENING AND DESIGN

(71) Applicant: Pfizer Inc., New York, NY (US)

(72) Inventors: Daniel Kurt GEHLHAAR, San Diego, CA (US); Daniel Janson MERMELSTEIN, San Diego, CA (US)

(73) Assignee: Pfizer Inc., New York, NY (US)

(21) Appl. No.: 19/427,481

(22) Filed: Dec. 19, 2025

Related U.S. Application Data
(60) Provisional application No. 63/736,706, filed on Dec. 20, 2024.

(51) Int. Cl. G16C 20/50; G16C 20/70

(52) U.S. Cl. CPC G16C 20/50; G16C 20/70

(57) ABSTRACT

Technology is disclosed for a method for predicting the bound conformation of a ligand in a protein binding site, the method includes selecting a plurality of model descriptors, calculating a docking probability for a ligand atom type occupying a particular point in a binding site using a neural network and converting the calculated probability to a numerical score suitable for ranking virtual screening poses using a weight matrix.

### CROSS REFERENCE TO RELATED APPLICATIONS

This application claims priority to U.S. Provisional Application No. 63/736,706 titled “Machine Learning Based 3D Virtual Screening Enrichment” filed on Dec. 20, 2024, of which has been incorporated by reference in its entirety.

### BACKGROUND OF THE INVENTION

It is common for a project team to look for novel chemical matter in early stages of the project. The team can start with a known receptor of known structure, either derived from X-ray crystallography, Cryo-EM, or homology modeling. They also start with some database of compounds, but these are either large commercial databases (up to billions of compounds) or internal collections in the millions of compounds. Projects usually only have resources to physically screen a few thousand to tens of thousands of compounds.

Virtual screening is important in early drug discoveries. By using computational methods to evaluate large chemical databases for potential active compounds, the process can significantly save time and costs during early drug development. However, traditional methods have limitations in accuracy and efficiency when they rely on docking algorithms and scoring functions. For example, traditional scoring features, based on physics models or numerical calculations, struggle with the complexity of protein ligand interactions, which can result in a high level of false positives and poor enrichment of active compounds.

It remains challenging to achieve high enrichment defined as retrieving a high proportion of active compounds from a predominantly inactive dataset. The Enrichment Factor at 1%, which is used to measure the improvement over random selection in recovering active compounds in the top 1% of results, can illustrate traditional methods' under-performance for advanced approaches.

Machine learning provides promising solutions by capturing complex patterns in protein-ligand interactions. However, many challenges remain when integrating machine learning into virtual screening, including the need for sophisticated and efficient computational power and algorithms for practical drug discovery applications and extensive data requirements. As such, there is a need for methods to predict protein binding efficiencies in an efficient and accurate manner.

### SUMMARY OF THE INVENTION

This summary is provided to introduce a selection of concepts in a simplified form that is further described below in the detailed description. This summary is intended neither to identify key features or essential features of the claimed subject matter nor to be used in isolation as an aid in determining the scope of the claimed subject matter.

Embodiments of the technologies described in the present disclosure enables technology for a method for predicting the bound conformation of a ligand in a protein binding site, the method includes selecting a plurality of model descriptors, calculating a docking probability for a ligand atom type occupying a particular point in a binding site using a neural network and converting the calculated probability to a numerical score suitable for ranking virtual screening poses using a weight matrix.

### BRIEF DESCRIPTION OF THE DRAWING

Aspects of the disclosure are described in detail below with reference to the attached figures, wherein:

FIG. 1 illustrates an exemplary descriptor triplet in accordance with the subject matter presented herein;

FIG. 2A illustrates exemplary positive data for the model in accordance with the subject matter presented herein;

FIG. 2B illustrates exemplary negative data for the model in accordance with the subject matter presented herein;

FIG. 3 illustratively depicts an exemplary cubic interpolated iso-surfaces on probability grids calculated on the binding site in accordance with the subject matter presented herein;

FIG. 4 illustrates a block diagram of a FitScore calculation in accordance with the subject matter presented herein;

FIG. 5 illustrates a procedure for tuning the weight matrix in accordance with the subject matter presented herein;

FIG. 6 illustrates a calculation of the Weighted Active Frequency in accordance with the subject matter presented herein; and

FIG. 7A and FIG. 7B illustrate compound designs using the virtual screening tool in accordance with the subject matter presented herein.

### DETAILED DESCRIPTION OF THE INVENTION

The subject matter of the present disclosure is described herein with specificity with the help of different aspects to meet statutory requirements. However, the description itself is not intended to limit the scope of this patent. The claimed subject matter might be embodied in other ways, including different steps or combinations of steps similar to the ones described in this present disclosure, in conjunction with other present or future technologies. Moreover, although the terms “step” and/or “block” may be used herein to connote different elements of methods employed, the terms should not be interpreted as implying any particular order among or between various steps disclosed herein, unless and except when the order of individual steps is explicitly stated. Each method described herein may comprise a computing process that may be performed using any combination of a hardware, firmware, and/or software. For instance, various functions may be carried out by a processor executing instructions stored in a computer memory. The methods may also be embodied as computer-useable instructions stored on computer storage media. The methods may be provided by a stand-alone application, a service or a hosted service (stand-alone or in combination with another hosted service), or a plug-in to another product, to name a few.

Aspects and embodiments of the present disclosure relate to aspects and/or embodiments of a method or scoring function, for predicting protein binding efficiencies; as well as virtual screening tools and compound design tools utilizing artificial intelligence and/or machine learning algorithms.

Docking, in the realm of computational chemistry, can be directed to as the prediction of the bound conformation of a flexible ligand in a protein binding site. In some ways docking algorithms can generate two distinct results, the orientation (or “pose”) of the ligand in the receptor, and a numerical score that indicates the quality of fit. In some instances, predicting the correct pose may be of high importance and may be the most emphasized metric for docking program performance. For example, it may be assumed that when a pose is significantly different than the actual binding mode (greater than 2.0 Å RMSD), the score cannot be trusted.

In a lead optimization context, involving docking of smaller libraries or a small number of proposed modifications to an existing lead, the poses themselves can be visualized and vetted manually. In this case the score of the ligand can be secondary to the pose, within a wide latitude. However, in a large-scale virtual screening context, involving millions of ligands, there is no practical way to visually inspect most of the poses, and the score can become of primary importance for ranking compounds and separating a small number of putative active compounds. This small number of active compounds can then be screened visually or rescored with more sophisticated computational methods, such as those utilizing advanced molecular mechanics. A typical virtual screen can consist of millions of compounds. Only a small subset of screened compounds can be realistically inspected or rescored. This number is generally several thousand compounds. In the context of million-compound docking runs, this means that generally 99% or more of docking results are typically discarded. Any “interesting” compounds will likely come in the top 5000-10000 hits (approximately 1% of the total results), and the rest will be ignored.

A useful metric for virtual screening enrichment is the Enrichment Factor at 1% (EF1). EF1 is the fold improvement over random in the recovery of actives in the top 1% of docking results. Theoretically, since at random you expect 1% of the actives to be returned for each 1% of the docking results, EF1 gives the percent of known actives recovered in the top 1% of docking results when sorted by score and thus has a maximum value of 100. This is generally true for large datasets and/or those with a very small fraction of known actives. For smaller datasets, or those with a larger fraction of known actives, EF1 can have a smaller maximum value; for example, for a dataset of 10000 compounds and 200 known actives, the maximum EF1 value is 50 (if the top 1% is all active, then those 100 are half of the total known actives, for an EF1 of 50).

In accordance with the subject matter disclosed herein, instead of using a new set of penalty and reward functions and the associated lengthy iterative process required to parameterize and tune such a set of heuristics, a plurality of databases of crystallographic protein-ligand complexes (CSDB) may be leveraged instead. In some embodiments, a machine learning model may be constructed to capture the complementarity data directly from crystal structures without having to choose and parameterize individual heuristic terms.

In accordance with the subject matter presented herein, a two-stage scoring function as presented herein (e.g., the FitScore) may be utilized to capture atomic complementarity in a numerical score, where the first stage can include a neural network trained on the CSDB data that predicts the probability of a particular ligand atom type occupying a particular point in the binding site; and a second stage uses the actual ligand atom types and locations (i.e., from a docked pose) along with their predicted probabilities from the neural network to generate a score via a tuned weight matrix. In some embodiments, the FitScore may be designed to be used in concert with existing physics- and heuristics-based scoring functions. Additionally, FitScore, using only a small number of atom types and distances, may be maximally interpretable both via the output grids and the underlying model parameters. In this way, FitScore effectively captures what human designers do when they evaluate docked poses.

### Model Descriptors

In some embodiments, ligand complementarity to a binding site may be based on the aggregate complementarity of every atom in the ligand and its respective protein environment, as defined by the location and orientation of binding site atoms near the ligand. A docked pose may be evaluated by looking at a small number of “nearby” atoms to the ligand and what interactions are being made. This can be modeled using an algorithm such as a neural network (e.g., a softmax output) to give a probability distribution for different predicted ligand atom types at a given receptor coordinate.

In practice, model descriptors may be chosen to match the intuitive approach taken by human designers. Atoms in molecular simulations may be assigned a “type” that generalizes the element and interactions that a particular atom can make; for example, whether an atom is aromatic, whether it is a hydrogen bond donor, whether it has a formal charge, etc. In some embodiments, one may start with a relatively large number of atom types, separating, for example, aliphatic and aromatic hydrogen bond donors and acceptors; and then merged types as one trained the model and found multiple types that converged to very similar trained parameters. The atom types used in the model may include: NONPOLAR, AROMATIC_NONPOLAR, DONOR, WEAK_DONOR, WEAK_DONOR_AROMATIC, CHARGED_DONOR, ACCEPTOR, CHARGED_ACCEPTOR, DONEPTOR, FLUORINE, and HALOGEN (all other halogens). In addition, a METAL type may be added for protein metal ions, and a WATER type for ordered water molecules, but these were not used as ligand atom types.

These atom types can be defined as follows. Acceptors include all oxygen, nitrogen, and sulfur atoms with lone pairs available for hydrogen bonding. Donors include all oxygen, nitrogen, and sulfur atoms with attached hydrogen atoms. Hydrogen bond donors and acceptors are considered charged (CHARGED_DONOR, CHARGED_ACCEPTOR) if they carry a formal charge in a standard resonant form (for example, both oxygens in an acid, or the three nitrogens in a guanidinium group). WEAK_ACCEPTOR is assigned to a specific subgroup of acceptors which includes ketone oxygen, sulfonamide/sulfone oxygen, and ether and thioether atoms. ACCEPTOR is used for all other acceptor atoms. DONOR is used for all donor atoms that are not charged. Atoms that are both donors and acceptors (for example, hydroxyl and terminal sulfonamide nitrogen) are classified as DONEPTOR. Carbon atoms with at least one hydrogen that are bonded to either oxygen or nitrogen, can be classified as WEAK_DONOR or WEAK_DONOR_AROMATIC, depending on whether the carbon is in an aromatic ring. All other atoms are classified as NONPOLAR or AROMATIC_NONPOLAR depending on whether they are in an aromatic ring. HALOGEN, FLUORINE, METAL, and WATER types are self-evident.

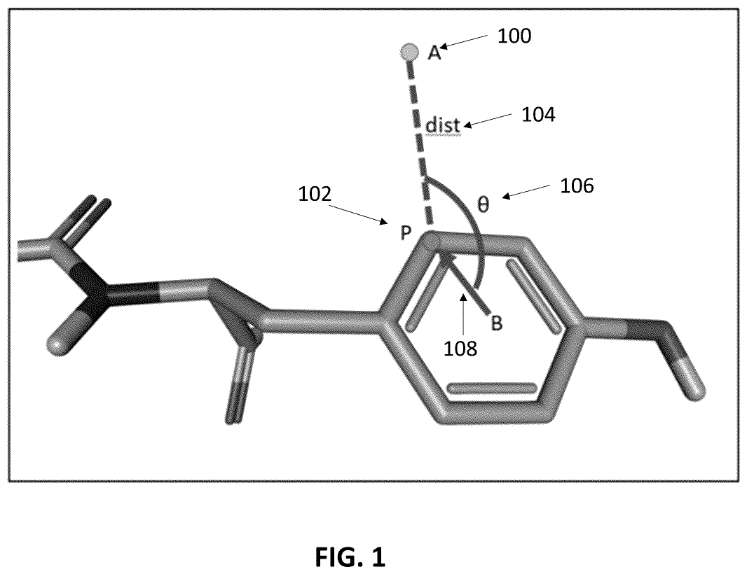

The descriptor for the model may consist of a vector of triplets (“descriptor triplets”), with each triplet consisting of the type, distance, and angle, referring to FIG. 1, from a point in the receptor to either a receptor heavy atom or hydrogen atom. As illustrated in FIG. 1, a descriptor triplet for binding site point A 100 and protein atom P 102 may consist of the type of atom P 102, the distance AP 104, and the angle APB 106. Where the vector BP 108 may be calculated as the sum of vectors from P 102 to its heavy atom neighbors, normalized to a unit vector, and then translated so its tail is at P. For atoms such as water and metal ions that lack heavy atom neighbors, this angle is set to zero. The type used for hydrogen atoms is the type of the bonded heavy atom. The descriptor vector consists of descriptor triplets for the nearest N receptor heavy atoms to the given binding site coordinate, ordered by distance, nearest to farthest, and then a similar set of triplets for the nearest N receptor hydrogen atoms. In some embodiments, models may include varying N between 3 and 20 and observed classification accuracy and model stability. In practice, the models having N>8 (e.g., N=10) were chosen for the production model.

Significant information may be captured in this descriptor vector, in a way that is insensitive to the translation and rotation of the bound complex. The “natural” information about nearby atoms and their relative orientations may be encoded; as well as some “implied” information such as the local curvature and solvent exposure (based on the distance distribution of the nearby heavy atoms). In some embodiments, no further “derived” data was explicitly added. No information about hydrogen bond distances or angles, electrostatics, pi stacking, halogen-based interactions, or any other such information was given explicitly to the model. In this fashion, the model the mimics the basic information available to a human designer—the atomic coordinates, types, and orientations- and the model is configured to discover its own “rules”.

### Model Data

In some embodiments, X-ray crystallographic protein-ligand complexes can be used as a source of information for the models described herein, for example, crystal structures of noncovalent protein-ligand with a resolution of 2.5 Å or better, followed by a filter to eliminate structures with severe steric clashes between protein and ligand. For example, 6000 prepared structures spanning a diverse set of protein families, including various proteases, kinases, phosphodiesterases, and receptors from crystal structure databases may be used. For structures containing multiple copies within the same asymmetric unit, only one copy was selected for the model, at random. In some embodiments, a model may be trained using a subset of two thousand plus structures that are publicly available.

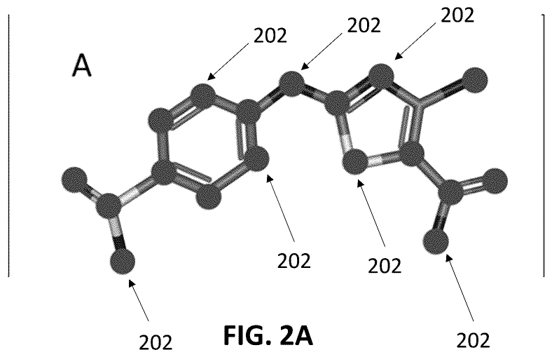

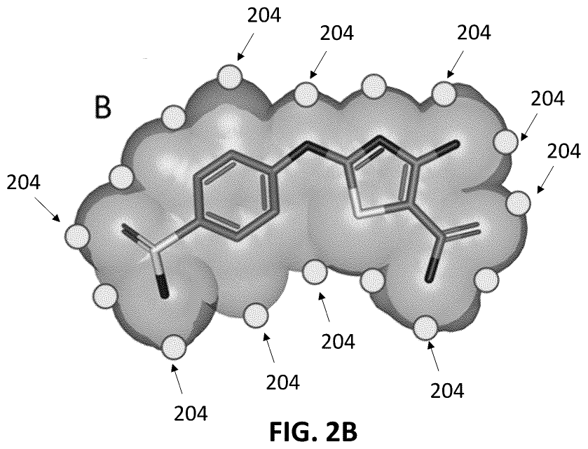

In practice, machine learning models may be fitted with both “positive” (desired) and “negative” (undesired) data to assist in discrimination and tuning. In a docking context, this translates to knowing where ligand atoms should not be placed in a receptor (negative), in addition to where they are known to be favorable (positive). For example, a pose with a severe close contact should be given a less favorable score than a pose with no close contacts. In traditional force fields, repulsive terms in the scoring function, such as van der Waals overlaps or electrostatic close approaches between same signed charges, account for this. In a machine learning model in accordance with the subject matter described herein, negative data may need to be provided explicitly. The positive data for the model are descriptors for the ligand heavy atoms, with the desired classification being the actual atom type at that location (a vector with unit probability for that type and zero probability for all other types), as illustrated in FIG. 2A, where positive data for the model, red dots 202, are sampled on atomic centers of known ligands. Negative data may be generated using a sparse sampling of surface points near the ligand as illustrated in FIG. 2B, where negative data for the model, yellow dots 204, may be sampled from the van der Waals surface of the known ligands. These negative points may be classified as one of two new types: NONE_CLOSE (for points that intersect at least one van der Waals radius of a protein atom) and NONE_FAR (for points that do not). These represent two distinct classes of negative data in a docking context. NONE_CLOSE are points that represent a van der Waals clash with the protein. NONE_FAR are locations where no ligand atoms are present such as at the solvent boundary or in an empty part of the receptor. These classifications would presumably be penalized less severely. The model was trained with an equal number of positive and negative data points.

### Model Training

In some embodiments, a classifier may be firstly built using the SciKit-Learn Random Forest Classifier framework using default parameters. When this model showed good classification results, validating that the chosen descriptors could yield reasonable classification accuracy, one may move onto the target framework with TensorFlow. A simple feed-forward neural network may be used due to its computational efficiency. Also, neural network models are compact and can be loaded very quickly. In some embodiments, the trained TensorFlow model can be several orders of magnitude smaller and faster than the Random Forest model while producing slightly higher classification accuracy. To avoid potential issues associated with using descriptors with discrete values, such as atom types, with neural network models, one may use a one-hot encoding of atom type data in the input feature vectors. This may result in a significant increase in the size of the input feature vectors, but no additional information was encoded.

In practice, the input data may be randomly divided into a training set with 95% of the data (N=123098) and a holdout/test set with 5% of the data (N=6479). In some embodiments, a model disclosed herein may be built with the Keras component of TensorFlow as a Sequential model, with two fully interconnected hidden layers of 60 and 40 nodes, followed by a softmax output layer. Hidden nodes used a rectified-linear activation function. All nodes used double-precision floating point representations. The model may be fitted using the Adam optimizer with an initial learning rate of 0.002, a batch size of 256, and 60 epochs. To minimize stalling, learning rate scheduling techniques used in training neural networks (e.g., a ReduceLROnPlateau callback) may be used to periodically reduce the learning rate when a loss function plateau was detected. The minimum learning rate may be set at 1.0e-5. This set of learning rate criteria may be adjusted to give a good balance between consistent convergence and prediction accuracy.

In some embodiments, model classifier types (the ligand atom types plus the two negative types) are not uniformly distributed in the input data. For example, half of the data consists of two types (NONE_CLOSE and NONE_FAR) while most of the remaining data consists of AROMATIC_NONPOLAR and NONPOLAR classes. Conversely, HALOGEN types may be rare. To account for this, weighting factors may be added to weight the different prediction classes inversely proportional to their frequency in the input data. It may initially appear that the distribution of predicted types (defined as the output type with the highest probability) do not match the distribution of known types in the input data. For example, HALOGEN may be overpredicted, while AROMATIC_NONPOLAR may be underpredicted. To rectify this, one may modify the weights of these types accordingly and refit the model. This process may be iterated until the predicted distribution of types significantly matched the distribution of the known atom type frequencies in the input data. The final weight scaling factors are shown in Table 1,

TABLE 1
Scale factors used for neural network
training based on input data point types.
Table 1: Scale factors used for neural network
training based on input data point types.

| | Unbiased | | Final |
|---|---|---|---|
| Type | Weight^{[a]} | Scale^{[b]} | Weight^{[c]} |
| NONPOLAR | 0.690 | 0.75 | 0.519 |
| AROMATIC_NONPOLAR | 0.346 | 0.89 | 0.307 |
| ACCEPTOR | 1.691 | 0.65 | 1.102 |
| CHARGED_ACCEPTOR | 5.530 | 0.16 | 0.876 |
| WEAK_ACCEPTOR | 2.840 | 0.40 | 1.138 |
| DONOR | 2.888 | 0.32 | 0.929 |
| WEAK_DONOR | 1.863 | 0.46 | 0.848 |
| WEAK_DONOR_AROMATIC | 4.042 | 0.27 | 1.095 |
| CHARGED_DONOR | 9.930 | 0.13 | 1.302 |
| DONEPTOR | 6.770 | 0.18 | 1.207 |
| FLUORINE | 10.031 | 0.13 | 1.321 |
| HALOGEN | 18.081 | 0.075 | 1.350 |
| NONE_CLOSE | 0.178 | 1.83 | 0.325 |
| NONE_FAR | 0.726 | 0.46 | 0.335 |

^{[a]}Unbiased weight determined by unbiased scaling from the proportion of present in the input training data.
^{[b]}Empirically determined scaling factors that minimized difference between predicted and actual type frequency.
^{[c]}The final scaling used in neural network training, the product of (A) and (B).

and the final predicted distributions for the test set are shown in Table 2.

TABLE 2
Distributions of actual training and predicted
test atom types in the final model.
Table 2: Distributions of actual training and
predicted test atom types in the final model.
The weight scale factors were tuned to make
these as similar as possible.

| | Fraction in | Predicted in |
|---|---|---|
| Type | Training Set | Test Set |
| NONPOLAR | 10.32% | 9.74% |
| AROMATIC_NONPOLAR | 20.68% | 21.64% |
| ACCEPTOR | 4.21% | 4.25% |
| CHARGED_ACCEPTOR | 1.30% | 1.13% |
| WEAK_ACCEPTOR | 2.51% | 2.45% |
| DONOR | 2.46% | 2.29% |
| WEAK_DONOR | 3.88% | 4.42% |
| WEAK_DONOR_AROMATIC | 1.76% | 2.01% |
| CHARGED_DONOR | 0.71% | 0.60% |
| DONEPTOR | 1.06% | 1.21% |
| FLUORINE | 0.70% | 1.08% |
| HALOGEN | 0.40% | 0.35% |
| NONE_CLOSE | 40.19% | 39.40% |
| NONE_FAR | 9.80% | 9.43% |

In some embodiments, the model's performance may be quite stable to changes in parameter values. The use of two hidden layers dramatically improved performance compared to a single hidden layer. In practice, the model parameters may be chosen to minimize the size of the model while maintaining classification accuracy. No additional hyperparameters were tuned beyond learning rate and model architecture as described above. In this model, the classification accuracy was 70.6% for the training set, and 66.3% for the test set. The model has 23474 tunable parameters, and was trained on 123098 data points, an over five-fold redundancy, minimizing the risk of overfitting.

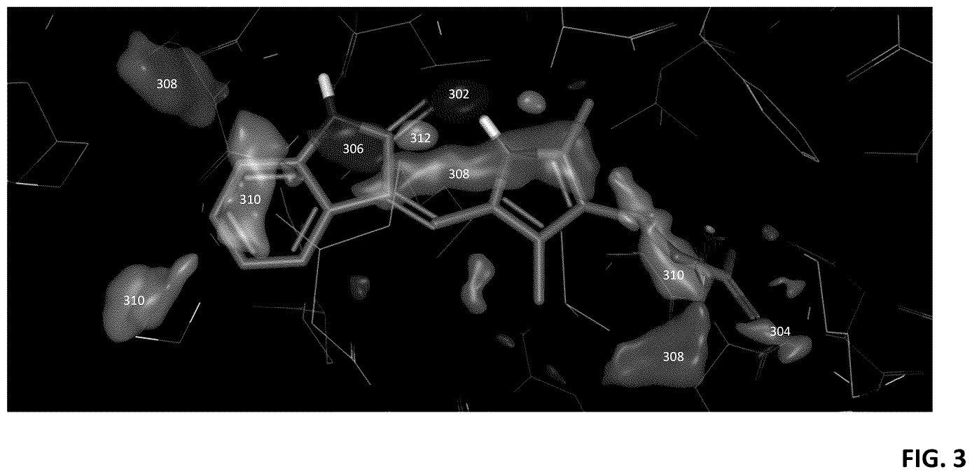

In practice, the neural network predictions can be visualized by calculating atom type probabilities on a uniform grid covering the binding site and then rendering isosurfaces of the resulting scalar fields. Referring now to FIG. 3, visualizing these scalar field isosurfaces can provide an intuitively interpretable view onto the data and allows quick verification that the probabilities generated by the model are “reasonable”. FIG. 3 illustrates cubic interpolated isosurfaces on probability grids calculated on the binding site of PDB entry 5YVC. The ligand was not present during the calculation and was not part of the training or test sets. Contours are for ACCEPTOR 302 (dark blue, 60% probability contour), CHARGED_ACCEPTOR 304 (light blue, 25% probability contour), DONOR 306 (red, 60% probability contour), AROMATIC_NONPOLAR 308 (dark green, 50% probability contour), NONPOLAR 310 (light green, 30% probability contour), and WEAK_DONOR_AROMATIC 312 (yellow, 40% probability contour). Note that key polar recognition sites are precisely and correctly localized, and there is distinct separation of aromatic and aliphatic nonpolar contours.

### Weight Matrix

In practice, the weight matrix may be used to convert the predicted probabilities from the neural network model to a numerical score suitable for ranking virtual screening poses. For example, the generated probabilities represent a concise fingerprint of the nature of the point in the binding site, and the weight matrix may attempt to quantify the “chemical intuition” that a structure-based designer might use to assess the “reasonableness” of a particular atom being placed at that location. In some embodiments, a hydrogen bond donor placed at a location suitable for a donor gets a large positive weight; and a hydrogen bond acceptor placed at that same location gets a large negative weight. Nonpolar atoms placed in that location would receive an intermediate weight. An example “toy” weight matrix is given in Table 3.

TABLE 3
Sample weight matrix with actual
ligand atom types and predicted types.
Table 3: A sample weight matrix with actual
ligand atom types (rows) and predicted types
(columns). For example, a DONOR placed where
expected gets a large positive weight, while
a DONOR placed where an ACCEPTOR is expected
gets a large negative weight. This matrix is
not necessarily symmetric. The effect of, for
example, placing a NONPOLAR where a HALOGEN
is expected, may not be scored the same as
placing a HALOGEN where a NONPOLAR is expected.
This can capture some important and subtle
chemistry and molecular recognition.

| | DONOR | ACCEPTOR | NONPOLAR | HALOGEN |
|---|---|---|---|---|
| DONOR | 2.0 | −2.0 | −1.0 | −0.5 |
| ACCEPTOR | −2.0 | 2.0 | −1.0 | 0.5 |
| NONPOLAR | −0.5 | −0.75 | 1.0 | 0.5 |
| HALOGEN | −1.0 | 0.75 | −0.25 | 1.5 |

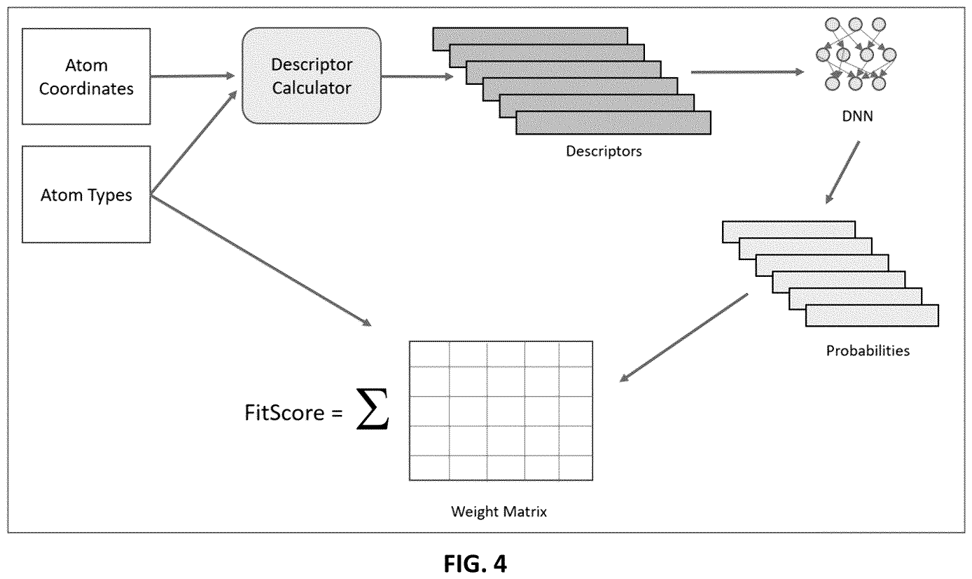

The FitScore may be calculated as the sum over all ligand heavy atoms of the scalar product between the neural network predicted probabilities of each atom, and the row of the weight matrix corresponding to that atom's known type. Referring now to FIG. 4, where the FitScore may be calculated by summing over all heavy atoms, the scalar (inner) product between probability vector generated by the neural network model for that atom, and the row of the weight matrix corresponding to the assigned atom type for that atom.

In some embodiments, the weight matrix may be optimized to maximize the difference in score distributions between a set of poses of known active ligands, and a corresponding set of inactive poses. The choice of “active” poses can be important, as a “poor” pose might mislead the tuning and emphasize an interaction that may not actually be important or desirable. A corresponding level of care may need to be taken when selecting the inactives compounds and poses used for the optimization. In some embodiments, a larger and more diverse set of inactives than of actives may be included in actual datasets.

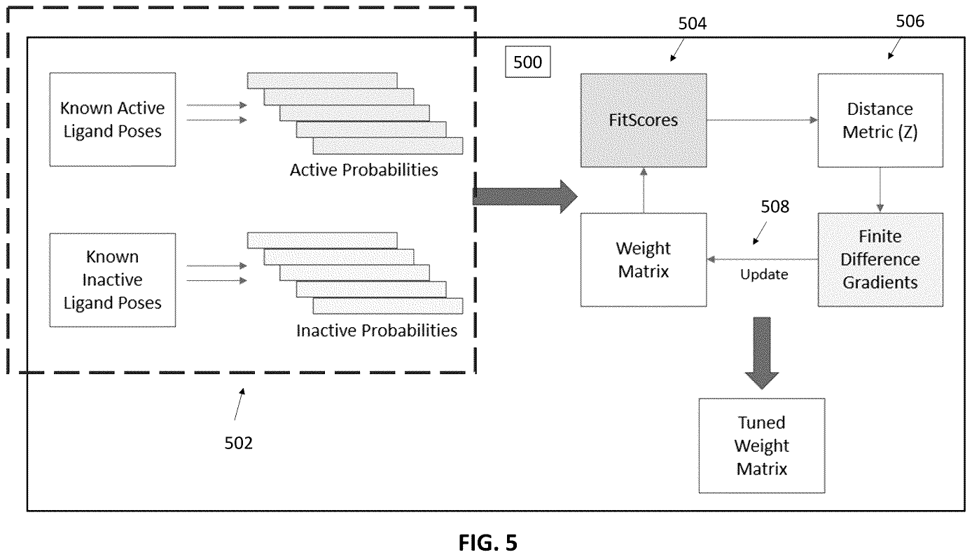

In practice, a 1:10 ratio of actives to inactives may be adopted for weight matrix optimization. Also, it should be appreciated that not all inactives are equal. Some may have relatively poor compatibility with the binding site and can be easily discriminated. In practice, one scoring function for a virtual screening enrichment can discriminate between true actives and those that are “almost actives”, that is, inactive ligands with a reasonable compatibility with the target binding site, that may otherwise get high scores in docking algorithms. Therefore, for FitScore tuning, one may sample inactives only from the best-scoring inactive poses, typically from the top 10% of returned poses for known inactive compounds. The process 500 for optimizing the weight matrix is illustrated in FIG. 5, where probability vectors may be pre-generated for selected sets of active and inactive ligands. These are then used to iteratively refine the weight matrix to maximize the distance between FitScore distributions for active and inactive ligands. In some embodiments, at step 502, a chosen set of active and inactive poses may be processed through the neural network to generate a probability vector for each ligand heavy atom. Subsequently, at step 504, the current weight matrix may be used to generate separate FitScore score distributions for the active and inactive poses. At step 506, the primary metric used for weight matrix optimization is the Z-metric, defined as the difference in the means of the active and inactive FitScore distributions divided by the square root of the sum of the squares of the standard deviations of the active and inactive FitScore distributions. The two-sample Kolmogorov-Smirnov (K-S) statistic may be used as a safeguard to ensure that the distributions are significantly distinct, that is, that the probability that the two distributions were actually sampled from the same underlying distribution is low. The central-difference estimate on each value of the weight matrix using a delta value of 1.0e-3. The step size along the gradient is then chosen using a golden section line search. The weight matrix is updated at step 508, and the tuning cycle is repeated until the search converges to a desired gradient magnitude, typically 1.0e-2, or until a given number of iterations has completed, typically 400.

In some embodiments, gradient searches may be inefficient compared to conjugate gradient or other higher-order searches. Gradient searches can also be prone to convergence to local minima. In this case, the search space appears very smooth, and the search routinely converges to very similar or identical solutions regardless of the starting values used in the weight matrix, within reasonable limits. In accordance with the subject matter presented herein, the weight matrix was seeded with all zero starting values. While it may have been more efficient to calculate analytical derivatives and use more complex minimization techniques, the direct gradient search converges fast enough to make these more complex methods unnecessary.

In some embodiments, one major criterion for choosing training sets for the weight matrix is ensuring that the cells of the weight matrix are “covered” by the training set, especially for known active compounds. While this is straightforward for common atom types, some care must be taken to ensure that rarer atom types, such as FLUORINE, HALOGEN, CHARGED_ACCEPTOR, and CHARGED_DONOR, are adequately represented in the training data. Ideally the training data should show instances where these atom types are routinely present in active compounds.

In some embodiments, the weight matrix must be constrained to achieve convergence. Without constraints, the gradient search will grow the absolute value of the weights without bound to maximize the difference in score distributions between active and inactive compounds. For this reason, one may arbitrarily clamp weight matrix values to be within +/−4.5. Other clamping values may be introduced for more subtle reasons. An optimization designed to push apart the scores for two distributions will apply a large scaling factor to a small scoring difference, where there might not be physical relevance, or for infrequent interactions. For example, docked poses rarely contain features such as close contacts, so weights related to NONE_CLOSE predictions tend to be unstable if not constrained. Since we know these should be repulsive interactions, we arbitrarily clamp these values to no higher than −0.5. NONE_FAR interactions should be of relatively little consequence, so they are clamped between −0.5 and 0.3. Molecular recognition for donors and acceptors should be of exceptional importance in protein-ligand interactions. However, examples of putting one class where the other is expected are rare in docking simulations, so these weights tend to be unstable. We clamp the weights for placing DONOR or CHARGED_DONOR atoms in locations predicted to contain ACCEPTOR or CHARGED_ACCEPTOR atoms, or vice versa, at a max weight of −2.0. Finally, we want to sufficiently penalize charged atoms in locations favoring nonpolar atoms, due to the severe associated desolvation penalties, so these interactions are clamped at a maximum value of −2.0, and the particularly egregious case of CHARGED_DONOR in a location predicted to contain CHARGED_ACCEPTOR, and vice versa, are clamped at the max penalty of −4.5.

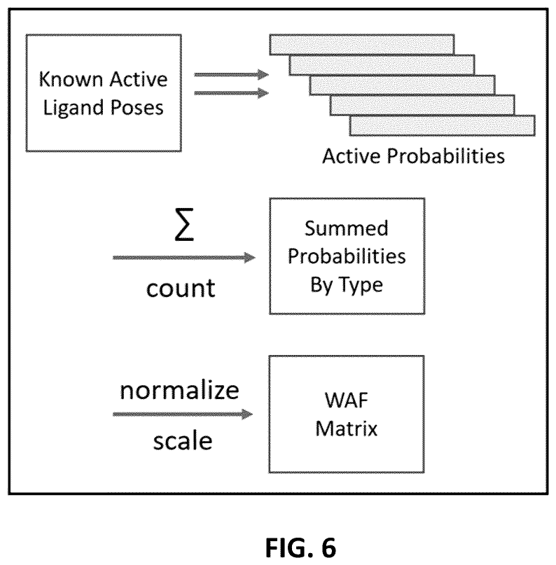

In some embodiments, the weight matrix may be relatively sparse in positive values. That is, in most active protein-ligand complexes, ligand atoms will be in regions that should be predicted to be quite likely to contain that, or a closely related, atom type. Most other combinations should be unfavorable. More generally, interactions that are infrequent or absent in the population of known actives, should be penalized. As such, as illustrated in FIG. 6, calculation of the Weighted Active Frequency may be performed by summing the calculated probabilities for the atoms in the active set, separated by atom type. These are then normalized by the count for that atom type, and then scaled by a factor of 100, to bring the values into a convenient range. In practice, one may cap the weight matrix elements with a WAF of 1.0 or lower, at a maximum value of −1.0, and cap the weight matrix elements with a WAF of 5.0 or lower, at a max value of 1.5.

### Virtual Screening

In practice, several datasets from the highly curated Directory of Useful Decoys, Enhanced (DUD-E) may be used for virtual screening enrichment validation. It should be appreciated that instead of molecular structures, only the docked poses and their resulting scores may be used to tune the weight matrix, to minimize potential bias risks.

In some embodiments, eight diverse validation cases from DUD-E were chosen: aldose reductase (ALDR), dihydrofolate reductase (DYR), estrogen receptor alpha (ESR1), tyrosine protein kinase LCK (LCK), cyclooxygenase-1 (PGH1), purine nucleoside phosphorylase (PNPH), tyrosine protein kinase SRC (SRC), and serine/threonine protein kinase WEE1 (WEE1). These represent a wide range of binding site shapes, sizes, and binding motifs, with structurally diverse known active compounds. In our experience, these span the range of difficulty from easy (WEE1, PNPH, ESR1), to medium (ALDR, DYR, LCK), to hard (PGH1, SRC), in terms of docking pose prediction and virtual screening enrichment predictions.

All ligands were downloaded from DUD-E in their SMILES representations. Tautomers, protomers, and 3-D coordinates were generated with the Schrodinger LigPrep tool, and output in SDF file format. An additional data tag was added to each molecule to indicate whether it was active or inactive, to simplify post-processing and data analysis. Protein and reference ligand structures were downloaded from DUD-E in PDB and MOL2 formats, respectively, and were processed with the Schrodinger PrepWizard using default settings. All ligands were docked into their target protein with GOLD using auto-scaling of the search parameters, the default scoring function (ChemPLP), the default number of docking runs, cavity detection based on the provided reference ligand for each target, and automated setting of protein and ligand atom types. For each ligand, the variant (tautomer, protomer, and dock) with the best ChemPLP score was kept, and all others were discarded, as would be typical in a large-scale virtual screening experiment.

For each target, the docked ligands may be divided randomly into two equal size sets, one for training, and one for testing. Subsequently, known active and inactive ligands in the training set may be used to tune the FitScore weight matrix. In some embodiments, random splits were used, but it should be appreciated that a more “time-series” approach could have been utilized as well for splitting the data, where all ligands generated before some date are used for training and all ligands generated after that date are used for testing. One procedure for choosing “actives” for FitScore tuning, as described above, entails careful examination of the binding poses, and only choosing those “known actives” that make “reasonable” or “likely favorable” interactions. In practice, this procedure could introduce unwanted bias which would be difficult to explain objectively, as such decisions are often made by experts based on their intuitions. Instead, the top 30 scoring known actives for each target were chosen as the “active” set, and then, following the procedure outlined previously, chose a random sampling of 300 inactives for each target from among those scoring in the top 20%. The result was 240 known actives and 2400 known inactives across the eight cases. These known actives and inactives were used to tune the FitScore weight matrix as described above. The final optimized weight matrix generated the statistics seen in Table 4.

TABLE 4
Metrics from the FitScore weight matrix tuning.
Table 4: Metrics from the FitScore weight matrix
tuning. FitScore distributions for active and
inactive poses are distinct, with the means
separated by several standard deviations..

| Metric | Result |
|---|---|
| Active FitScore Mean | 11.42 |
| Active FitScore Stdev | 3.58 |
| Inactive FitScore Mean | 2.99 |
| Inactive FitScore Stdev | 3.66 |
| Z-Metric | 1.65 |
| K-S Statistic | 0.748 |
| K-S Probability | <1.0e−15 |

In some embodiments, one may use this weight matrix to calculate the FitScore for all ligands in the test set for all targets. For each target, the enrichment factor may be calculated at 1% (EF1) using both the GOLD ChemPLP score, and the FitScore.

The Enrichment Factors at 1% (EF1) for the test set poses, using the ChemPLP and FitScore scoring functions, are given in Table 5. In most cases, FitScore yields similar or better results than ChemPLP.

TABLE 5
Virtual screening enrichment results.
Table 5: Virtual screening enrichment results
for the test cases, calculated as the
Enrichment Factor at 1% (EF1) for the
ChemPLP and FitScore scoring functions.

| Test Case | ChemPLP EF1 | FitScore EF1 |
|---|---|---|
| ALDR | 12.3 | 8.2 |
| DYR | 14.8 | 33.2 |
| ESR1 | 27.8 | 21.9 |
| LCK | 7.2 | 23.5 |
| PGH1 | 7.2 | 0.9 |
| PNPH | 19.6 | 28.4 |
| SRC | 1.0 | 14.6 |
| WEE1 | 54.0 | 47.0 |

Furthermore, the FitScore also provides strong “early recognition”, i.e. that even within the top 1% of compounds, as many of the active compounds are ranked near the top as possible. In some embodiments, the Boltzmann Enhanced Discrimination of the Receiver Operator Characteristic (BEDROC) may be calculated to quantify the early recognition power of FitScore at EF 1%. BEDROC scores are in line with what would be expected given the EF 1% values in table 6.

TABLE 6
Virtual screening BEDROC score.
Table 6: Virtual screening BEDROC scores
for the test cases, calculated for the
top 1% scoring compounds.for the ChemPLP
and FitScore scoring functions.

| | ChemPLP | FitScore |
|---|---|---|
| Test Case | BEDROC | BEDROC |
| ALDR | 0.255 | 0.243 |
| DYR | 0.228 | 0.486 |
| ESR1 | 0.495 | 0.382 |
| LCK | 0.122 | 0.388 |
| PGH1 | 0.121 | 0.045 |
| PNPH | 0.300 | 0.461 |
| SRC | 0.025 | 0.294 |
| WEE1 | 0.876 | 0.897 |

This indicates that the top 1% of compounds is not “backloaded”, and that there are a reasonable number of actives that score at the very top of the output.

As presented herein, the FitScore can generate high degrees of enrichment, even in what is perhaps the most difficult use case, where (1) the scoring is completely detached from the pose generation process; and (2) the known active poses were chosen using a different scoring function and without detailed manual screening. Human designers have repeatedly shown the ability to iteratively optimize small molecules using structure-based drug design. However, capturing the rules used in that decision-making process has proven elusive. Instead, in accordance with the embodiments presented herein, a very small and physically intuitive set of descriptors were used, similar to those used by human designers, and then the “rules” were extracted using machine learning in a way that can be intuitively scrutinized and evaluated. It should be appreciated that the FitScore model was able to deduce the desired interactions, based solely on exposure to a large and diverse dataset.

In some other embodiments, the FitScore probabilities generated by the neural network can also be represented visually as scalar field isosurfaces (FIG. 3) for direct inspection and use by design chemists. For example, if an element in the weight matrix optimizes to a value that seems counterintuitive, this can point to issues with descriptors such as atom type. Indeed, our decision to split WEAK_DONOR and WEAK_DONOR_AROMATIC into two separate atom types, was partially based on weight matrix optimized values that did not agree with our intuitions and examples commonly found in high-resolution crystal structures.

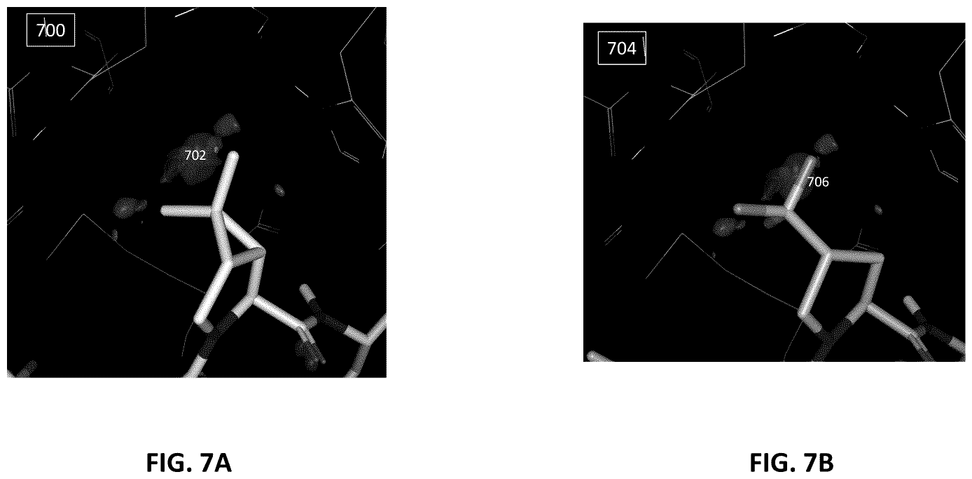

In practice, a screening or design tool such as the FitScore presented herein can be used to predict structure of compounds in the binding site and assess the goodness of fit, which in turn may directly contribute to the design and production of novel compounds. In some embodiments, the machine learning model in FitScore can generate a computed probability (based on its training data) that a given atom type (e.g. donor, acceptor, halogen) is likely to be a “good match” for a particular part of a binding site, thereby guiding the design of a compound. These probabilities can be visualized as “blobs of likely goodness” in our molecular modeling software and can (and does) guide compound design. For example, referring now to FIG. 7A, illustrated is an image of a part of a binding site from a crystal structure of a novel compound 700. The purple blobs 702 show the areas where FitScore suggests fluorine would be favorable (at least 30% likelihood). These appear just here and in a very small number of other places in the binding site. Referring now to FIG. 7B, which illustrates a crystal structure of a follow-on compound 704, where the complicated ring structure was replaced by a trifluoromethyl group 706. This compound 704 has much better binding affinity and properties than does compound 700. As such, a tool such as the FitScore, as used herein, may be utilized to provide compound design to a human designer to optimize and/or design novel compounds.

Many different arrangements of the various components depicted, as well as components not shown, are possible without departing from the scope of the claims below. Embodiments of the disclosure have been described with the intent to be illustrative rather than restrictive. Alternative embodiments will become apparent to readers of this disclosure after and because of reading it. Alternative means of implementing the aforementioned can be completed without departing from the scope of the claims below. Certain features and sub-combinations are of utility and may be employed without reference to other features and sub-combinations and are contemplated within the scope of the claims.

What is claimed is:

1. A method for predicting the bound conformation of a ligand in a protein binding site, the method comprising:
selecting a plurality of model descriptors;
calculating a docking probability for a ligand atom type occupying a particular point in a binding site using a neural network; and
converting the calculated probability to a numerical score suitable for ranking virtual screening poses using a weight matrix.

2. The method of claim 1, wherein the plurality of model descriptors comprises Acceptor descriptors and Donor descriptors.

3. The method of claim 2, wherein the Acceptor descriptors comprise oxygen, nitrogen, and sulfur atoms with lone pairs available for hydrogen bonding.

4. The method of claim 2, wherein the Donor descriptors comprise oxygen, nitrogen, and sulfur atoms with attached hydrogen atoms.

5. The method of claim 1 further comprising calculating atom type probabilities on a uniform grid covering the binding site.

6. The method of claim 5 further comprising rendering isosurfaces of the resulting scalar fields.

7. The method of claim 1 further comprising optimizing the weight matrix by maximizing the difference in score distributions between a set of poses of known active ligands, and a corresponding set of inactive poses.

8. The method of claim 7, where in the ratio of actives to inactives is 1:10.

9. The method of claim 7 further comprising processing a set of active and inactive poses through the neural network to generate a probability vector.

10. The method of claim 7, wherein the primary metric used for weight matrix optimization is the difference in the means of the active and inactives.

11. The method of claim 1 further comprising building a classifier using random forest classifier with default parameters.
